(19) United States
(12) Patent Application Publication
(10) Pub. No.: US 2010/0179416 A1
(43) Pub. Date: Jul. 15, 2010

(54) Medical Systems and Methods

(76) Inventors: Michael Hoey, Shoreview, MN (US); John H. Shadduck, Tiburon, CA (US)

Correspondence Address: SHAY GLENN LLP, 2755 CAMPUS DRIVE, SUITE 210, SAN MATEO, CA, 94403, US

(21) Appl. No.: 12/687,734

(22) Filed: Jan. 14, 2010

Related U.S. Application Data
(60) Provisional application No. 61/144,658, filed on Jan. 14, 2009.

(51) Int. Cl. A61B 5/055; A61B 8/00; A61B 18/04; A61M 31/00; A61B 18/02

(52) U.S. Cl. 600/411; 600/439; 606/27; 604/93.01; 606/21

(57) ABSTRACT

A prostate therapy system is provided that may include any of a number of features. One feature of the prostate therapy system is that it can access a prostate lobe transrectally. Another feature of the prostate therapy system is that it can image the prostate lobe transrectally. One feature of the prostate therapy system is that it can deliver condensable vapor into the prostate to ablate the prostate tissue. Methods associated with use of the prostate therapy system are also covered.

### CROSS REFERENCE TO RELATED APPLICATIONS

This application claims the benefit under 35 U.S.C. 119 of U.S. Provisional Patent Application No. 61/144,658, filed Jan. 14, 2009, titled “Medical Systems and Methods.” This application is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present invention relates to an apparatus and a related method for treatment of a prostate disorder in a human male using a minimally invasive trans-rectal approach.

### BACKGROUND OF THE INVENTION

Several systems and methods have been developed or proposed for the treatment of prostate tissue to alleviate BPH symptoms or to treat prostate tissue. For example, tissue ablation methods have been based on RF ablation, microwave ablation, high intensity focused ultrasound (HIFU), cryoablation, radiation, surgery, and brachytherapy. Surgical methods with and without robotic assistance have been developed for removal of diseased prostate tissue.

The apparatus, techniques and methods disclosed herein are adapted to for the treatment of prostate tissue in general and more particularly are focused on treatment of BPH (benign prostatic hyperplasia) and prostate cancer. BPH is a common problem experienced by men over about 50 years old that relates to urinary tract obstruction. Prostatic hyperplasia or enlargement of the prostate gland leads to compression and obstruction of the urethra which results in symptoms such as the need for frequent urination, a decrease in urinary flow, nocturia and discomfort.

Ablation of prostatic tissue with electromagnetic energy is well known and has the advantage of allowing a less invasive approach. For example, high-frequency current in a electrosurgical ablation or prostatic tissue causes cell disruption and cell death. Tissue resorption by the body's wound healing response then can result in a volumetric reduction of tissue that may be causing urinary tract obstruction. One disadvantage or high-frequency current of laser ablation is potential tissue carbonization that results in an increased inflammatory response and far longer time to heal following the ablation.

### SUMMARY OF THE INVENTION

A method of providing a treatment of prostatic tissue in a human male patient comprises positioning a transrectal introducer assembly in the patient, the assembly including a flow channel having an open termination in a tool working end, actuating an imaging system within the introducer to image the prostate, extending the tool working end to a targeted region of the prostate under imaging guidance, and delivering flow media through the flow channel into the targeted region to treat the targeted region.

In some embodiments, the imaging system comprises transrectal ultrasound. In other embodiments, the imaging system comprises endorectal MRI.

In some embodiments, the flow media is a high temperature condensable vapor. In another embodiment, the flow media includes a drug. In one embodiment, the flow media includes at least one of an anesthetic, an anti-inflammatory agent, an anti-fungal agent, and an antibiotic agent.

In one embodiment, the method further comprises condensing the vapor to apply energy to the targeted region.

In some embodiments, the tool working end is advanced manually. In another embodiment, the tool working end is advanced at least in part by a spring mechanism. The tool working end can be advanced a predetermined distance relative to the assembly, for example.

In one embodiment, the tool working end delivers the flow media from a single outlet. In another embodiment, the tool working end delivers the flow media from a plurality of outlets.

In one embodiment, the tool working end delivers a cryogenic flow media.

In one embodiment, the method further comprises extending the tool working end into a plurality of targeted regions under imaging guidance and delivering flow media to each of said targeted regions.

Another method of treating prostatic tissue in a human male patient is provided, comprising imaging prostatic tissue with a transrectal ablation and imaging system, obtaining biopsy cores from a plurality of targeted regions of the prostate utilizing the transrectal ablation and imaging system under the imaging guidance, determining whether said biopsy cores include a neoplastic cell, and delivering ablative energy through the transrectal ablation and imaging system to ablate prostatic tissue having neoplastic cells.

In some embodiments, the ablative energy is delivered by a high temperature condensable vapor. In other embodiments, the ablative energy is delivered by a liquid or fluid. In another embodiment, the ablative energy is delivered by a gas.

In some embodiments, the ablative energy freezes tissue of the targeted regions. In other embodiments, the ablative energy heats the targeted regions.

In one embodiment, the ablative energy is delivered for between 1 second and 300 seconds.

In one embodiment, a prostate cancer ablative therapy system is provided comprising an access assembly configured for transrectal positioning adjacent a patient prostate, an imaging system carried by the access assembly and configured to image the prostate, a tool extendable from the access assembly and configured to extend into the prostate, and a vapor delivery mechanism configured to deliver condensable vapor through the tool into the prostate to apply ablative energy to the prostate.

In some embodiments, the imaging system comprises transrectal ultrasound. In other embodiments, the imaging system comprises endorectal MRI.

In some embodiments, the tool comprises a needle.

In one embodiment, the vapor delivery mechanism delivers high temperature condensable vapor. The vapor can be configured to have a temperature of approximately 60° C. to 100° C.

In one embodiment, the system further comprises a computer controller configured to deliver vapor for an interval ranging from 0.1 second to 30 seconds.

In another embodiment, the system further comprises a source of a pharmacologic agent for delivery with the vapor.

### BRIEF DESCRIPTION OF THE DRAWINGS

FIG. 1 is a vapor energy delivery system and more particularly a cut-away view of a handle portion of an instrument with an inductive heating assembly for applying vaporization energy to a fluid flow together with a looped flow system for maintaining a circulating flow of high energy vapor which is releasable on demand to flow through an extension member to interact with tissue.

FIG. 2 is a schematic view of the inductive heating assembly of FIG. 1.

FIG. 3 is a schematic view of a sectional view of a patient's prostate and accessing the prostate with a tool working end guided by a trans-rectal ultrasound imaging system.

FIG. 4 is a sectional view of a patient prostate showing multiple biopsy locations in a systematic prostate cancer diagnosis with each biopsy location comprising a potential treatment location.

FIG. 5 is another sectional view of a patient prostate showing the potential multiple biopsy and treatment locations.

### DETAILED DESCRIPTION OF THE INVENTION

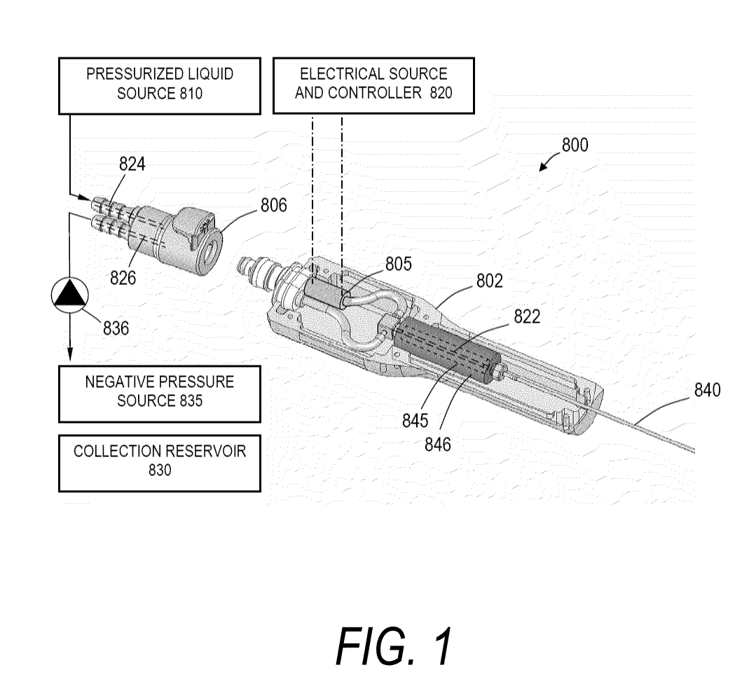

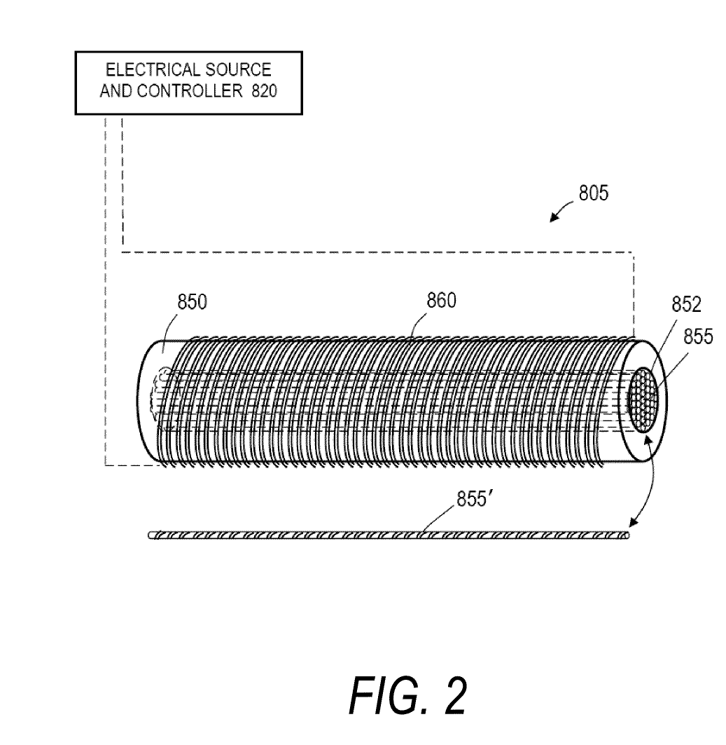

Referring to FIGS. 1 and 2, one embodiment of the invention is shown that includes a probe 800 with handle member 802 that is coupled to an elongated axial extension member 840 having a suitable length and diameter form ranging from 2 to 8 mm that can be configured for introduction into a patient's urethra or prostate, or accessing prostatic tissue trans-rectally or endoscopically. The system is configured to deliver a heated vapor, for example water vapor, to tissue as described in the following co-pending U.S. Patent Applications: U.S. patent applications Ser. No. 10/681,625 filed Oct. 7, 2003 titled “Medical Instruments and Techniques for Thermally-Mediated Therapies”; Ser. No. 11/158,930 filed Jun. 22, 2005 titled “Medical Instruments and Techniques for Treating Pulmonary Disorders”; Ser. No. 11/244,329 (Docket No. S-TT-00200A) filed Oct. 5, 2005 titled “Medical Instruments and Methods of Use” and Ser. No. 11/329,381 (Docket No. S-TT-00300A) filed Jan. 10, 2006 titled “Medical Instrument and Method of Use”. All of the above applications are incorporated herein by this reference and made a part of this specification, together with the specifications of all other commonly-invented applications cited in the above applications.

The generation and delivery of a collapsible, high energy vapor for various therapeutic procedures is further disclosed in systems with ‘remote” vapor generation systems or sources in co-pending Provisional Application Nos. 60/929,632; 61/066,396; 61/068,049, or with vapor generator in a handle or working end, or combination thereof, as described in Provisional Application Nos. 61/068,130; 61/123,384; 61/123,412; 61/126,651; 61/126,612; 61/126,636; and 61/126,620, all of which are incorporated herein by reference in their entirely.

FIG. 1 illustrates another vapor generation system 800 in a handle 802 of elongated introducer which comprises and inductive heating system similar to that described in Provisional Application Nos. 61/123,416; 61/123,417; and 61/126,647. In FIG. 1, the handle 802 is coupled by temperature resistant fitting 806 to a fluid source 810 that delivers liquid at a controlled flow rate and pressure. The liquid flow passes through a heat emitter or applicator 805 that comprises an inductive heater coupled to an electrical source and controller indicated at 820. The system and handle is configured for a looped liquid/vapor flow to provide vapor to working end or exit channel 822 to deliver the vapor to a tissue site. The system has inflow channel indicated at 824 and outflow channel at 826 that can communicate with a collection reservoir 830 and/or a negative pressure source 835. A valve 836, for example, operated by a footswitch is provided in outflow channel 826 to re-direct vapor into the outflow channel 822 and extension member 840. A vapor generation system 800 as shown in FIG. 1 can be used for any surgical/medical application, with the extension member 840 comprising a needle, an elongate probe or flexible catheter and the like. This system can be used for a catheter for delivering energy for endovascular applications, for treating respiratory tract disorders, for endometrial ablation treatments or for needle ablation treatments. In the embodiment of FIG. 1, an optional secondary heater 845 is shown with a concentric insulator 846. This secondary heater can add further vaporization energy to vapor that starts to flow through channel 822. The secondary heater can be an inductive heater or a resistive heater that uses a microporous material to provide a large surface area to apply energy to the vapor to remove any water droplets. This system can provide a vapor that is at least 90% water vapor. The secondary heater is operatively coupled to the electrical source and controller 820 by electrical leads (not shown).

FIG. 2 illustrates a vapor generating inductive heater 805 that in on embodiment comprises a ceramic cylinder 850 about 1.0″ to 1.5″ in length and 0.25″ in diameter with a 0.10″ bore 852 therein. The bore is packed with a plurality of small diameter hypotubes 855 of a 316 stainless steel that is magnetic responsive. In one embodiment, the hypotubes 855 are 0.016 thin wall tubes. A winding 860 of one to ten layers having and an axial length of about 1.0″ is provided about the cylinder 850 for inductive heating of the tubes 855 using very high frequency current from an electrical source. In one embodiment the winding 860 can be 26 Ga. Copper wire with a Teflon coating. It has been found that delivering at least 50 W, 100 W, 200 W, 300 W, or 400 W with suitable flow rates of water can produce very high quality vapor, for example 90% vapor and better. In FIG. 2, it can be seen that an inductively heated hypotube 855′ also can be spiral cut to provide flexibility for such an inductive heater to be positioned in a catheter or probe working end. For example, such flexible heatable elements can be carried in the bore of a flexible high temperature resistant polymeric insulative member such to provide a flexible catheter that is configured for endovascular navigation. An insulation layer about an exterior of the inductive heater is not shown. In general, the inductive system 800 can configured to provide a high quality vapor media with precise parameters in terms of vapor quality, exit vapor pressure from a working end, exit vapor temperature, and maintenance of the parameters within a tight range over a treatment interval. All these parameters can be controlled with a high level of precision to achieve controlled dosimetry, no matter whether the particular treatment calls for very low pressures (e.g., 1-5 psi) over a treatment interval or very high pressures (200 psi or greater) and no matter whether the treatment interval is in the 1-10 second range or 2 to 5 minute range.

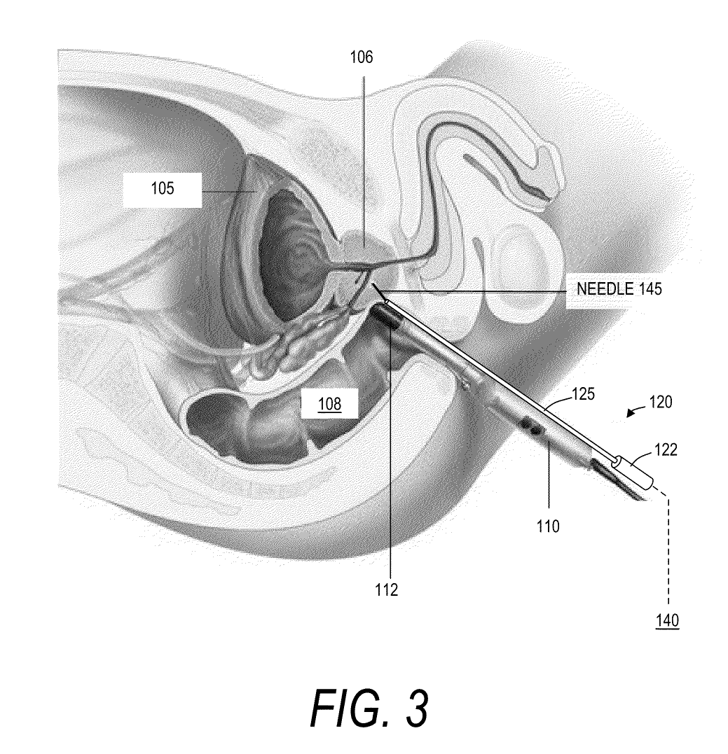

Referring now to FIG. 3, a Transrectal ultrasound (TRUS) guided needle ablation of prostatic tissue system is shown. In FIG. 3, a system 120 is depicted schematically that can be used for localized ablation of prostate tissue, or for ablation of lobe of a prostate. In FIG. 3, the system 120 can include an introducer member 110, an ultrasound probe 112, a sleeve assembly 125, and a needle 145. The needle 145 can include a lumen in fluid communication with a high temperature condensable vapor source 140. The distal end of the needle can also include an outlet or a plurality of outlets configured to deliver high temperature condensable vapor from the vapor source to tissue. The system 120 of FIG. 3 is illustrated in relation to the appropriate anatomy, such as the bladder 105 and the colon 108. The system can further comprise a computer controller configured to deliver vapor for an interval of time.

In current practice, practically all prostate cancers are diagnosed by means of systematic TRUS-guided prostate biopsy with a biopsy needle in an approach indicated in FIG. 3. A biopsy needle, such as an 18-gauge needle, is typically used. Many physicians perform one to three biopsies on palpable lesions and further need biopsies on additional lesions viewed by ultrasound. The biopsy procedure also may be performed utilizing endorectal MRI.

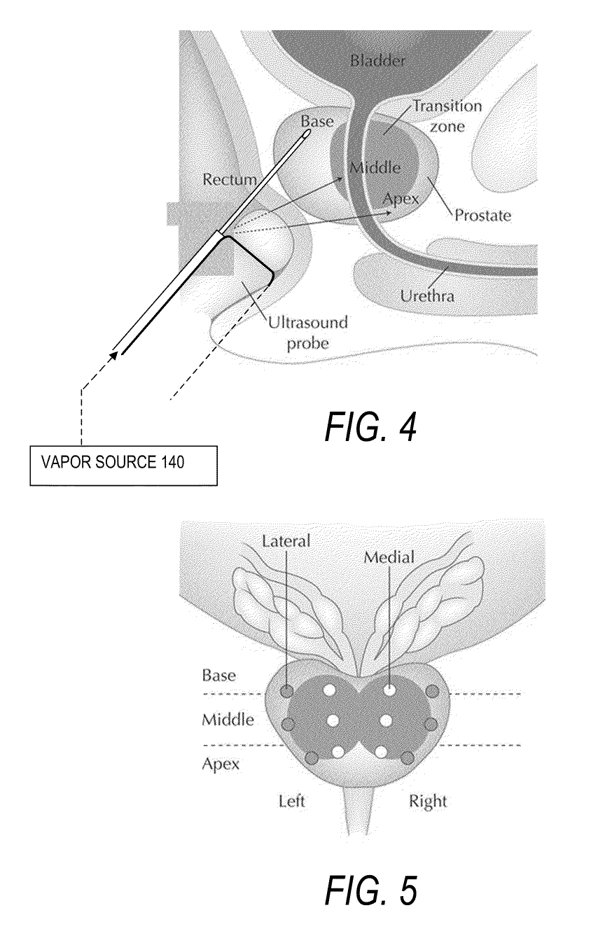

In another method, a larger number of biopsy cores are taken systematically from the peripheral zone (see FIGS. 4 and 5) for example medially and laterally from the apex, middle, and base of the prostate on each side together with biopsy cores from the transition zone. Such a strategy can provide significant information about the location and extent of prostate cancer. The information gained from this biopsies then can be used for treatment planning, such planning to preserve, resect or ablate all or part of certain regions of the prostate to provide treatment margins. Studies have shown a correlation of the quantity of cancer in systematic biopsy specimens (expressed as the number of positive cores, percentage of positive cores, total percentage of cancer in cores, or ratio of cancer length to total core length) with the grade of cancer. Further, the percentage of positive biopsy cores has been reported to be a significant predictor of prostate cancer mortality.

In one method, a system adapted for biopsies can be used for needle ablation of selected regions of the prostate that are determined by biopsy to have neoplastic growth. The TRUS systems now allow for collection of biopsy cores and return to the same prostate location with a follow-up ablative procedure. Thus, selected localized regions of the prostate can be treated in am minimally invasive procedure that can be an office-based procedure.

In the embodiment shown in FIG. 3, a prostate cancer ablative therapy system 120 is depicted which comprises an introducer member 110 or access assembly configured for transrectal positioning with the working end adapted for positioning adjacent to the patient's prostate 106. A transrectal ultrasound probe 112 can be used for this purpose and a sleeve assembly 125 can be assembled with the TRUS system for extending a sharp tool or needle 145 to a selected depth into the prostate. The tool can extend from about 5 mm to 500 mm and can be manually insertable or can be spring-loaded. The extent to which the tool can be extended can be a predetermined distance. The system further includes a vapor delivery mechanism 122 configured to deliver condensable vapor from a source 140 through the tool or needle 145 into the prostate to apply ablative energy to the prostate (see FIGS. 3 and 4).

Another embodiment of the system can incorporate an endorectal MRI mechanism rather than an ultrasound probe.

In one embodiment, the system includes a vapor delivery mechanism that delivers water vapor. The system can utilize a vapor source configured to provide vapor having a temperature of at least 60° C., 70° C., 80° C., 90° C. or 100° C.

In one embodiment, the system further comprises a computer controller configured to deliver vapor for an interval ranging from 0.1 second to 30 seconds. In other embodiments, the vapor can be delivered from between 1 and 300 seconds.

In one embodiment, the needle working end carries a plurality of vapor outlets for diffusing vapor propagation in the prostate tissue.

In another embodiment, the system further comprises a source of a pharmacologic agent for delivery with the vapor.

In another embodiment, the system can deliver ablative energy to the prostate tissue by delivering cryogenic flow media from a cryogenic fluid delivery mechanism to freeze tissue, or from a working end carrying at least one RF electrode, or by at least one light fiber within the tool working end for applying ablative light energy to the prostate.

As can be understood from FIGS. 3 and 4, the needle 145 can be straight or curved and keyed with its housing to penetrate into tissue in a selected configuration.

In general, a method of providing a treatment for ablating prostatic tissue in a human male patient, and comprises positioning a transrectal introducer assembly in the patient, wherein the assembly includes a flow channel having an open termination in a tool working end. Another step of the method comprises actuating an imaging means within the introducer to image the prostate, and extending the tool or needle working end 145 to a targeted region of the prostate under imaging guidance. Thereafter, the method includes delivering flow media through the flow channel into the targeted region to treat the targeted region. In one method, the flow media comprises a high temperature condensable vapor that applies ablative energy upon condensation to the targeted neoplastic region. In another aspect of the method, the system can be used to deliver a flow media including at least one of an anesthetic, an anti-inflammatory agent, an anti-fungal agent, and an antibiotic agent.

In another method of the invention, the tool or needle working end can be advanced manually or at least in part by a spring mechanism.

In general, the methods of the invention include delivery of a condensable vapor that undergoes a phase change to provide applied energy of at least 250 cal/gm, 300 cal/gm, 350 cal/gm, 400 cal/gm and 450 cal/gm of the vapor.

In another method of the invention, the apparatus and method depicted in FIGS. 3-4 can be used for globally ablating tissue in at least one prostate lobe to treat prostate cancer.

In another method of the invention, the apparatus and method depicted in FIGS. 3-4 can be used for ablating prostate tissue, or volumetrically removing tissue, in a treatment of BPH. In one embodiment, the system can comprises an access assembly configured for transrectal positioning adjacent a patient prostate, imaging means carried by the access assembly for imaging the prostate, a tool extendable from the assembly for extending into the prostate, and tissue removal means carried by the tool working end to volumetrically remove prostate tissue for reducing pressure on the urethra. Additionally, the system can include an energy source and thermal energy emitter for sealing margins of the removed tissue, such as a source of condensable vapor, an RF source, a resistive heater, or a light source.

In another aspect of the invention, the treatment with vapor can be monitored during treatment ultrasound. In one method, the introduction of vapor can be imaged utilizing a transrectal ultrasound system commercialized by Envisioneering Medical Technologies.

In another aspect of the invention, the system may contemporaneously be used to deliver fluids to targeted locations in the prostate for medical purposes, such as for general or localized drug delivery, chemotherapy, or injections of other agents that may be activated by vapor or heat.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

What is claimed is:

1. A method of providing a treatment of prostatic tissue in a human male patient, comprising:
positioning a transrectal introducer assembly in the patient, the assembly including a flow channel having an open termination in a tool working end;
actuating an imaging system within the introducer to image the prostate;
extending the tool working end to a targeted region of the prostate under imaging guidance; and
delivering flow media through the flow channel into the targeted region to treat the targeted region.

2. The method of claim 1 wherein the imaging system comprises transrectal ultrasound.

3. The method of claim 1 wherein the imaging system comprises endorectal MRI.

4. The method of claim 1 wherein the flow media is a high temperature condensable vapor.

5. The method of claim 4 further comprising condensing the vapor to apply energy to the targeted region.

6. The method of claim 1 wherein the flow media includes a drug.

7. The method of claim 1 wherein the flow media includes at least one of an anesthetic, an anti-inflammatory agent, an anti-fungal agent, and an antibiotic agent.

8. The method of claim 1 wherein the tool working end is advanced manually.

9. The method of claim 1 wherein the tool working end is advanced at least in part by a spring mechanism.

10. The method of claim 1 wherein the tool working end is advanced a predetermined distance relative to the assembly.

11. The method of claim 1 wherein the tool working end delivers the flow media from a single outlet.

12. The method of claim 1 wherein the tool working end delivers the flow media from a plurality of outlets.

13. The method of claim 1 wherein the tool working end delivers a cryogenic flow media.

14. The method of claim 1 further comprising extending the tool working end into a plurality of targeted regions under imaging guidance and delivering flow media to each of said targeted regions.

15. A method of treating prostatic tissue in a human male patient, comprising:
imaging prostatic tissue with a transrectal ablation and imaging system;
obtaining biopsy cores from a plurality of targeted regions of the prostate utilizing the transrectal ablation and imaging system under the imaging guidance;
determining whether said biopsy cores include a neoplastic cell; and
delivering ablative energy through the transrectal ablation and imaging system to ablate prostatic tissue having neoplastic cells.

16. The method of claim 15 wherein the ablative energy is delivered by a high temperature condensable vapor.

17. The method of claim 15 wherein the ablative energy is delivered by a fluid.

18. The method of claim 15 wherein the ablative energy is delivered by a gas.

19. The method of claim 15 wherein the ablative energy freezes tissue of the targeted regions.

20. The method of claim 15 wherein the ablative energy heats the targeted regions.

21. The method of claim 15 wherein the ablative energy is delivered for between 1 second and 300 seconds.

22. A prostate cancer ablative therapy system comprising:
an access assembly configured for transrectal positioning adjacent a patient prostate;
an imaging system carried by the access assembly and configured to image the prostate;
a tool extendable from the access assembly and configured to extend into the prostate; and
a vapor delivery mechanism configured to deliver condensable vapor through the tool into the prostate to apply ablative energy to the prostate.

23. The system of claim 22 wherein the imaging system comprises transrectal ultrasound.

24. The system of claim 22 wherein the imaging system comprises endorectal MRI.

25. The system of claim 22 wherein the tool comprises a needle.

26. The system of claim 22 wherein the vapor delivery mechanism delivers high temperature condensable vapor.

27. The system of claim 22 wherein the vapor is configured to have a temperature of approximately 60° C. to 100° C.

28. The system of claim 22 further comprising a computer controller configured to deliver vapor for an interval ranging from 0.1 second to 30 seconds.

29. The system of claim 22 further comprising a source of a pharmacologic agent for delivery with the vapor.
